(19) United States
(12) Patent Application Publication
(10) Pub. No.: US 2023/0157876 A1
(43) Pub. Date: May 25, 2023

(54) VITREORETINAL VISUALIZATION FOR OPHTHALMIC PROCEDURES

(71) Applicant: Alcon Inc., Fribourg (CH)

(72) Inventor: Steven T. Charles, Memphis, TN (US)

(21) Appl. No.: 17/937,675

(22) Filed: Oct. 3, 2022

Related U.S. Application Data
(60) Provisional application No. 63/281,293, filed on Nov. 19, 2021.

(51) Int. Cl. A61F 9/008; G02B 27/48; A61B 3/13

(52) U.S. Cl. CPC A61F 9/008; G02B 27/48; A61B 3/13; A61F 2009/00863

(57) ABSTRACT

In certain embodiments, an ophthalmic system for visualizing an interior of an eye includes an illumination system and a visualization system. The illumination system illuminates the interior of the eye. The illumination system includes an annular illuminator that directs annular illumination, which has an illumination axis, towards the interior of the eye. The visualization system provides an image of the interior of the eye. The visualization system comprises visualization optical elements, which include an objective lens and oculars. The objective lens receives light reflected from the interior of the eye. The oculars, which have an ocular axis, transmit the reflected light to yield an image of the interior of the eye. In other embodiments, the illumination system comprises a multi-beam illuminator that directs multiple illumination beams towards the interior of the eye.

### TECHNICAL FIELD

The present disclosure relates generally to ophthalmic systems, and more particularly to vitreoretinal visualization for ophthalmic procedures.

### BACKGROUND

Vitreoretinal eye procedures are performed in the vitreoretinal region of the eye. Examples of such procedures include: breaking up vitreous clumped pre-existing collagen fibers (“floaters”); vitreous traction of a flap tear (“horseshoe tear”) before in-office pneumatic retinopexy for limited retinal detachments; residual vitreoretinal traction after surgical vitrectomy; residual retinal tissue causing retinal detachment (or elevation) due to incomplete surgical retinectomy; selected small diabetic traction retinal detachments; and selected vitreomacular traction syndrome cases.

A doctor must be able to see the vitreoretinal region in order to successfully perform a procedure. Moreover, appropriate illumination is key to effective vitreoretinal visualization. Unfortunately, in some situations, known systems fail to provide illumination that yields effective visualization.

### BRIEF SUMMARY

In certain embodiments, an ophthalmic system for visualizing an interior of an eye includes an illumination system and a visualization system. The illumination system illuminates the interior of the eye. The illumination system includes an annular illuminator that directs annular illumination, which has an illumination axis, towards the interior of the eye. The visualization system provides an image of the interior of the eye. The visualization system comprises visualization optical elements, which include an objective lens and oculars. The objective lens receives light reflected from the interior of the eye. The oculars, which have an ocular axis, transmit the reflected light to yield an image of the interior of the eye.

Embodiments may include none, one, some, or all of the following features.

The ophthalmic system includes a laser device that directs a treatment laser beam towards the interior of the eye.

The annular illuminator includes a laser source and annular optical elements. The laser source provides illumination light, and the annular optical elements modify the illumination light to yield the annular illumination. The laser source may provide the illumination light as a laser beam with a speckle pattern. The annular optical elements may include first and second axicons, where the first axicon transforms the illumination light into an annular distribution of light, and the second axicon modifies the annular distribution of light to yield the annular illumination. The annular optical elements may include an axicon and a spherical lens, where the axicon transforms the illumination light into an annular distribution of light, and the spherical lens modifies the annular distribution of light to yield the annular illumination. The annular optical elements may include an achromatic lens that focuses the annular illumination.

The annular illuminator includes a laser source and a spatial light modulator. The laser source provides an illumination light, and the spatial light modulator modifies the illumination light to yield the annular illumination.

The annular illuminator comprises an illumination ring, which includes lights disposed about the illumination ring.

The illumination axis is substantially coincident with the ocular axis.

The illumination axis is at an angle to the ocular axis.

The system comprises a slit lamp microscope.

In certain embodiments, an ophthalmic system for visualizing an interior of an eye includes an illumination system and a visualization system. The illumination system illuminates the interior of the eye. The illumination system comprises a multi-beam illuminator that directs illumination beams, which have an illumination axis, towards the interior of the eye. The multi-beam illuminator includes a laser source and one or more multi-beam optical elements. The laser source provides illumination light, and the multi-beam optical elements modify the illumination light to yield the illumination beams. The visualization system provides an image of the interior of the eye. The visualization system comprises visualization optical elements, which include an objective lens and oculars. The objective lens receives light reflected from the interior of the eye. The oculars, which have an ocular axis, transmit the reflected light to yield an image of the interior of the eye.

Embodiments may include none, one, some, or all of the following features.

The ophthalmic system includes a laser device that directs a treatment laser beam towards the interior of the eye.

The one or more multi-beam optical elements comprise a lenslet array that transforms the illumination light into the illumination beams.

The illumination beams are arranged into a circular pattern.

The illumination beams are arranged into a rectangular pattern.

The laser source provides a laser beam with a speckle pattern.

The illumination axis is substantially coincident with the ocular axis.

The illumination axis is at an angle to the ocular axis.

The system comprises a slit lamp microscope.

In certain embodiments, an ophthalmic system for visualizing an interior of an eye includes an illumination system, a visualization system, and a laser device. The illumination system illuminates the interior of the eye. The illumination system includes an annular illuminator that directs annular illumination, which has an illumination axis, towards the interior of the eye. The visualization system provides an image of the interior of the eye. The visualization system comprises a slit lamp microscope that has visualization optical elements, which include an objective lens and oculars. The objective lens receives light reflected from the interior of the eye. The oculars, which have an ocular axis, transmit the reflected light to yield an image of the interior of the eye. The laser device directs a treatment laser beam towards the interior of the eye. In the embodiments, the annular illuminator includes: a laser source that provides illumination light with a speckle pattern and annular optical elements that modify the illumination light to yield the annular illumination, the optical elements comprising a first axicon that transforms the illumination light into an annular distribution of light, a second axicon or a spherical lens that modifies the annular distribution of light to yield the annular illumination, and an achromatic lens that focuses the annular illumination; or a laser source that provides an illumination light and a spatial light modulator that modifies the illumination light to yield the annular illumination; or an illumination ring comprising lights disposed about the illumination ring.

In certain embodiments, an ophthalmic system for visualizing an interior of an eye includes an illumination system, a visualization system, and a laser device. The illumination system illuminates the interior of the eye. The illumination system comprises a multi-beam illuminator that directs illumination beams, which have an illumination axis, towards the interior of the eye. The multi-beam illuminator includes a laser source and one or more multi-beam optical elements. The laser source provides illumination light with a speckle pattern, and the multi-beam optical elements modify the illumination light to yield the illumination beams, which are arranged into a circular pattern or a rectangular pattern. The multi-beam optical elements comprise a lenslet array that transforms the illumination light into the illumination beams. The visualization system provides an image of the interior of the eye. The visualization system comprises a slit lamp microscope with visualization optical elements, which include an objective lens and oculars. The objective lens receives light reflected from the interior of the eye. The oculars, which have an ocular axis, transmit the reflected light to yield an image of the interior of the eye. The laser device directs a treatment laser beam towards the interior of the eye.

### BRIEF DESCRIPTION OF THE DRAWINGS

FIG. 1 illustrates an example of an ophthalmic system that provides vitreoretinal visualization for ophthalmic procedures, according to certain embodiments;

FIGS. 2 and 3 illustrate examples of illumination systems, according to certain embodiments, where FIG. 2 illustrates an example of a coaxial illumination system and

FIG. 3 illustrates an example of an angled illumination system;

FIG. 4 illustrates an example of an ophthalmic laser system that may utilize an illumination system described herein, according to certain embodiments;

FIGS. 5A through 5C illustrate examples of annular illuminators, according to certain embodiments, where FIG. 5A illustrates an annular illuminator with one or more axicons, FIG. 5B illustrates an annular illuminator with an illumination ring, and FIG. 5C illustrates an annular illuminator with a spatial light modulator (SLM);

FIGS. 6A and 6B illustrate examples of multi-beam illuminators, according to certain embodiments, where FIG. 6A illustrates a multi-beam illuminator implemented as an angled illumination system, and FIG. 6B illustrates a multi-beam illuminator implemented as a coaxial illumination system; and

FIG. 7 illustrates an example of a method for providing vitreoretinal visualization for ophthalmic procedures that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Vitreoretinal visualization (i.e., visualization of the vitreous and/or retina) can be difficult because some targets, such as eye floaters, are almost transparent and absorb very little light. In addition, external illumination of the vitreoretinal area is limited by Purkinje images, which are reflections from the surfaces of the cornea and lens. Moreover, laser vitreoretinal procedures are typically real-time, see-aim-and-shoot procedures, so visualization should be in real-time, stereo, and in color. For example, when treating eye floaters, the doctor should have real-time visualization to see movement of the floaters in response to laser shots. In addition, the doctor should be able to see the lens and retina in stereo and in color, as they provide anatomic landmarks that prevent spatial disorientation.

These and other challenges render known vitreoretinal imaging techniques unsatisfactory in certain situations. Accordingly, certain embodiments presented here provide real-time, stereo, and color vitreoretinal visualization. The embodiments use different types of illumination, which can be implemented in a variety of ways, to enhance vitreous visualization.

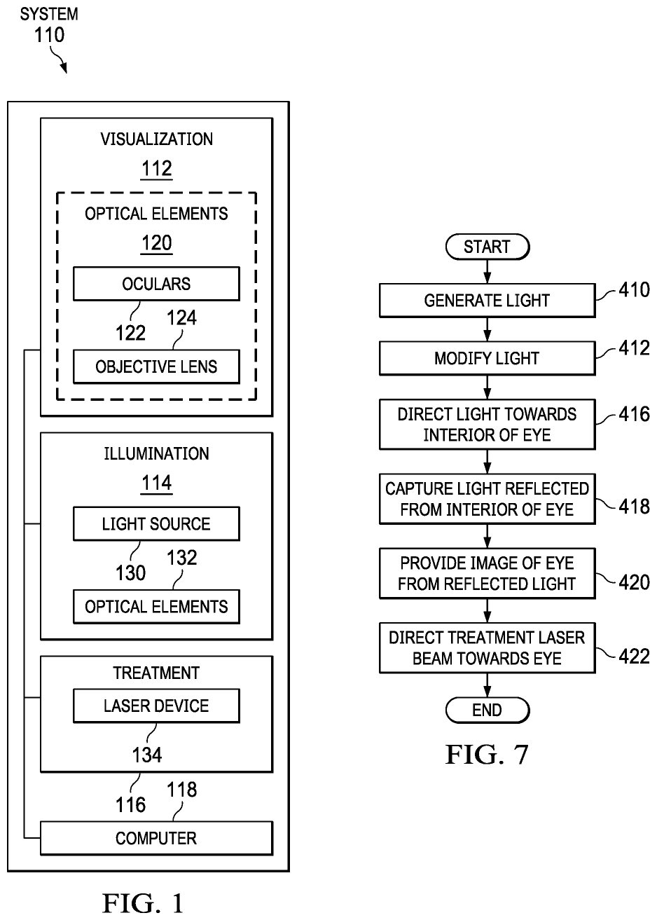

FIG. 1 illustrates an example of an ophthalmic system 110 that provides vitreoretinal visualization for ophthalmic procedures, according to certain embodiments. Ophthalmic system 110 illuminates the interior of the eye in order to provide an enhanced image of the interior, such as the vitreoretinal region. The vitreoretinal region comprises at least a portion of the vitreous and/or the retina.

As an overview of system 110, in the example, ophthalmic system 110 includes a visualization system 112, an illumination system 114, a treatment system 116, and a computer 118. Visualization system 112 includes optical elements 120, such as oculars 122 and an objective lens 124. Illumination system 114 includes a light source 130 and optical elements 132. Treatment system 116 includes a laser device 134.

Turning to the details of system 110, visualization system 112 receives light reflected from an eye and provides an image of the interior of the eye from the reflected light. Optical elements 120 modify the light reflected from the eye to yield an image of the eye. Optical elements 120 may be included in a slit lamp stereo microscope. In general, an optical element is a component that can act on (e.g., transmit, reflect, refract, diffract, collimate, condition, shape, focus, modulate, and/or otherwise act on) light. Examples of optical elements include a lens, a lens array, a mirror, a prism, a diffraction grating, a spatial light modulator (SLM), and a polarizer.

Continuing with visualization system 112, objective lens 124 collects and focuses the reflected light to yield an image of the eye, and oculars 122 magnify the image. Oculars 122 typically have left and right view paths with an ocular axis that coincides with a middle path midway between the left and right view paths.

Illumination system 114 provides light to illuminate at least a part of the vitreoretinal region, e.g., the vitreous and/or retina. Light source 130 generates illumination light, e.g., an illumination laser beam. In certain embodiments, light source 130 provides the illumination light as an illumination laser beam with an intrinsic speckle pattern. Light source 130 may be a laser beam source. Optical elements 132 modify the illumination light to yield any suitable illumination, e.g., one or more of the following types of illumination, and direct the illumination light along an illumination path towards the eye. The types of illumination include the following:

(1) Annular Illumination (AN): Annular illumination is light (e.g., white light-emitting diode (LED) light) provided as a tube or a hollow cone (such as a truncated cone), where light absent from the interior. Annular illumination has an axis, e.g., the axis of the tube or cone of illumination. If the axis of the annular illumination is substantially coincident with an axis of the eye (e.g., visual or optical axis), retinal reflections and Purkinje images may be reduced.

(2) Multi-Beam Illumination (MB): Multi-beam illumination is light provided as a plurality of light beams, e.g., a plurality of laser beams. Multi-beam illumination has an axis, e.g., an axis substantially in the center of the pattern of beams and parallel to the beams. Multi-beam illumination enhances visualization of targets, e.g., vitreous floaters.

(3) Speckle Pattern (SP): The mutual interference of a set of coherent wavefronts of light (such as laser light) produce a speckle pattern. The speckle pattern enhances visualization of vitreous of targets, e.g., vitreous floaters. The speckle pattern may be used with any suitable optical configuration, e.g., a single beam, a slit beam, and/or multiple beams. For example, non-pulsed dual aiming beams may have intrinsic speckle. The beams may be co-aligned and focused on the same image plane as the stereo microscope, illumination light, and treatment laser beam.

The types of illumination may be implemented in any suitable manner, and an implementation may have any suitable type of illumination, e.g., only Speckle Pattern SP, only Annular Illumination AN, only Multi-Beam Illumination MB, or any combination of SP, AN, and MB, such as SP and AN or SP and MB. Examples of implementations include the following.

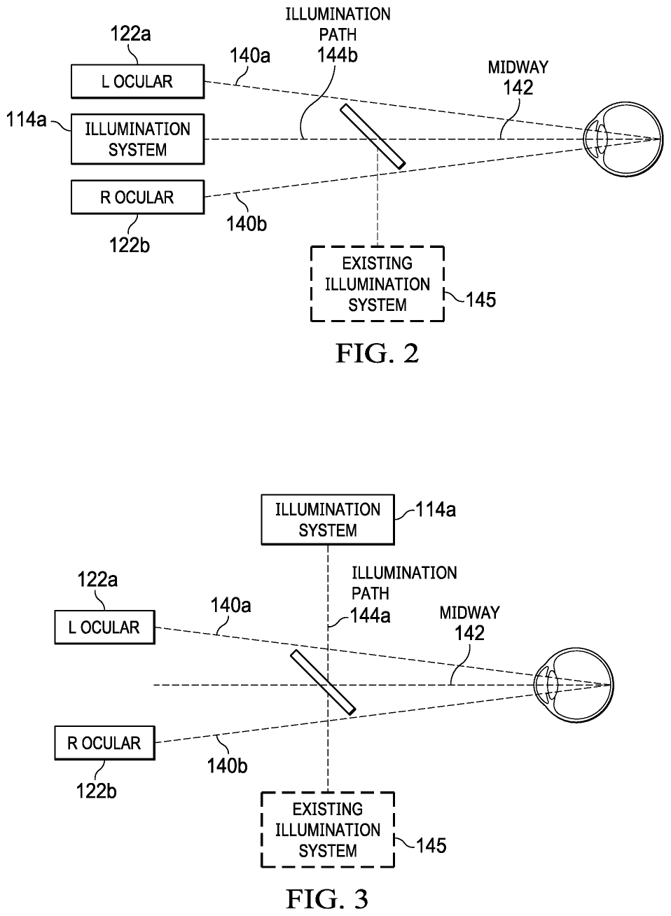

(1) Coaxial Implementation: From the output of illumination system 114, the light travels on an illumination path between the left and right view paths of oculars 122 such that the illumination path is coincident with (e.g., within +/−3 or 5 degrees of) the midway path. Illumination system 114 or at least the output of light may be located between the left and right view paths of oculars 122. A coaxial illumination system 114 is described in more detail with reference to FIG. 2.

(2) Angled (or Oblique) Implementation: At the output of illumination system 114, the light travels on an illumination path that is at an angle (e.g., greater than 3 or 5 degrees, such as 45 to 135 degrees or 70 to 110 degrees) to the midway path. An optical element, such as a mirror or beam splitter, directs the light to be coincident with the midway path. An angled illumination system may be used as the main illumination system or may augment the main illumination system using a beam splitter. An angled illumination system 114 is described in more detail with reference to FIG. 3.

Treatment system 116 includes laser device 134 that provides a treatment laser beam to treat the eye. Laser device 134 may include any suitable laser, e.g., a nanosecond, femtosecond, or picosecond laser with any suitable gain medium (e.g., Yb-doped fiber laser). The laser beam may have any suitable wavelength, e.g., in a range from 500 nm to 1100 nm. Any suitable repetition rate may be used, e.g., 3 Hz to MHz, and any suitable pulse energy may be used, e.g., an energy level sufficient to yield plasma in the eye tissue. Computer 118 provides instructions to systems 112, 114, 116 to perform visualization procedures.

FIGS. 2 and 3 illustrate examples of illumination systems 114 (114a and 114b), according to certain embodiments. In the examples, oculars 122 include left L ocular 122a and right R ocular 122b, with left L view path 140a and right R view path 140b, respectively. A midway path 142 is located midway between left L view path 140a and right R view path 140b. When system 110 is used to view an eye, midway path 142 is typically substantially coincident with an axis of the eye. Each illumination system 114 (114a and 114b) has an illumination path 144 (144a and 144b), respectively. In certain embodiments, illumination system 114 may supplement an existing or additional illumination system 145. In other embodiments, illumination system 114 may be the main illumination system.

FIG. 2 illustrates an example of a coaxial illumination system 114a. In the example, illumination system 114 is located between left L view path 140a and right R view path 140b such that illumination path 144a is coincident with midway path 142. Since the illumination and viewing paths are separated, the illumination source cannot be seen by the view path.

FIG. 3 illustrates an example of an angled illumination system 114b. In the example, the light output of illumination system 114 is not located on midway path 142. An optical element 146 directs the illumination light onto midway path 142.

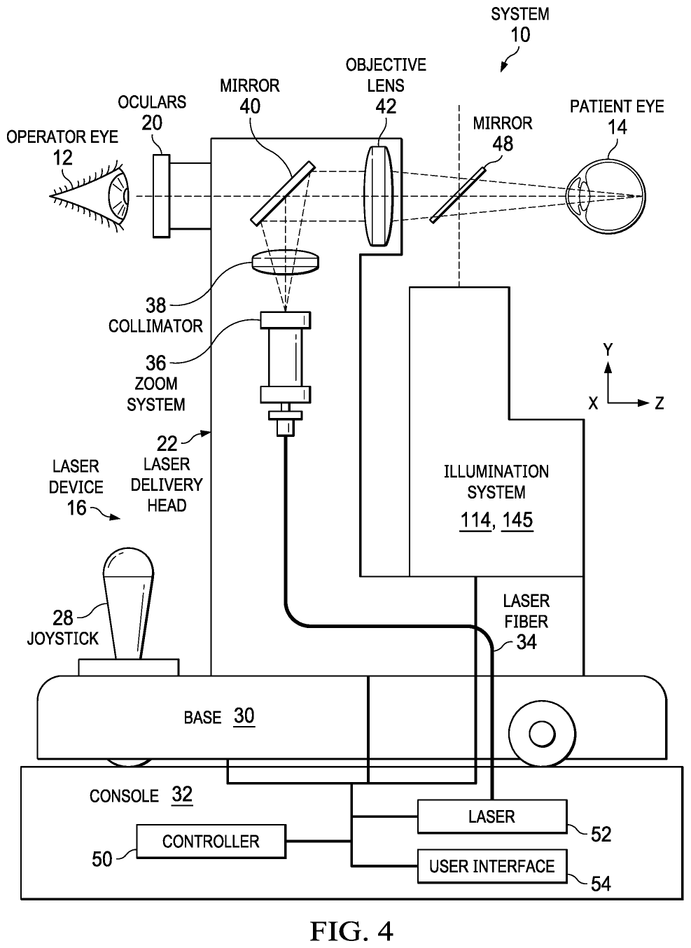

FIG. 4 illustrates an example of an ophthalmic laser system 10 that may utilize an illumination system 114, according to certain embodiments. In the example, ophthalmic laser system 10 comprises oculars 20, a laser delivery head 22, an illumination system 114, 145, a positioning device (such as a joystick 28), a base 30, and a console 32, coupled as shown. Laser delivery head 22 includes a laser fiber 34, a zoom system 36, a collimator 38, a mirror 40, and an objective lens 42, coupled as shown. Console 32 includes a computer (such as a controller 50), a laser 52, and a user interface 54, coupled as shown.

As an overview, ophthalmic laser system 10 includes a laser device 16 (e.g., laser 52, laser fiber 34, and laser delivery head 22) and a viewing portion (e.g., oculars 20, objective lens 42, mirror 48, and illumination system 114, 145). Operator eye 12 utilizes the optical path from oculars 20 through mirror 40, objective lens 42, and mirror 48 to view patient eye 14. A laser beam follows the laser path from laser 52 through laser delivery head 22 and mirror 48 to treat patient eye 14. According to the overview, laser device 16 directs a laser beam comprising laser pulses towards a target within eye 14. The viewing portion gathers light reflected from within eye 14 to yield an image of eye 14. Controller 50 instructs laser device 16 to direct the laser pulses towards the target.

In more detail, in certain embodiments, oculars 20 allow operator eye 12 to view patient eye 14. Laser delivery head 22 delivers a laser beam of laser pulses from laser 52 of console 32 to patient eye 14. Laser fiber 34 of delivery head 22 transports the laser beam from laser 52 to the end of fiber 34. Zoom system 36 includes optical elements that change the spot size of the laser beam that exits fiber 34. Collimator 38 collimates the laser beam, and mirror 40 directs the beam through objective lens 42, which focuses the beam. Zoom system 36 and collimator 38 are configured to direct a parallel laser beam to mirror 40, in order to focus the laser beam onto the image plane of the viewing portion. Mirror 40 may be a dichroic mirror that is reflective for the laser beam wavelength and transmissive for visible light.

Illumination system 114, 145 may be an illumination system 114 as described herein or an existing illumination system 145 (e.g., a slit illuminator) to be supplemented by an illumination system 114 as shown in FIGS. 2 and 3. Base 30 supports laser delivery head 22 and illumination systems 114, 145. Joystick 28 moves base 30 in the x-, y-, and z-directions. Console 32 includes components that support the operation of system 10. Controller 50 of console 32 controls of the operation of components of system 10, e.g., base 30, laser delivery head 22, illumination systems 114, 145, laser 52, and/or user interface 54. Laser 52 supplies the laser beam. Laser 52 of laser device 16 may be similar to the laser of laser device 134 of FIG. 1. User interface 54 communicates information between the operator and system 10.

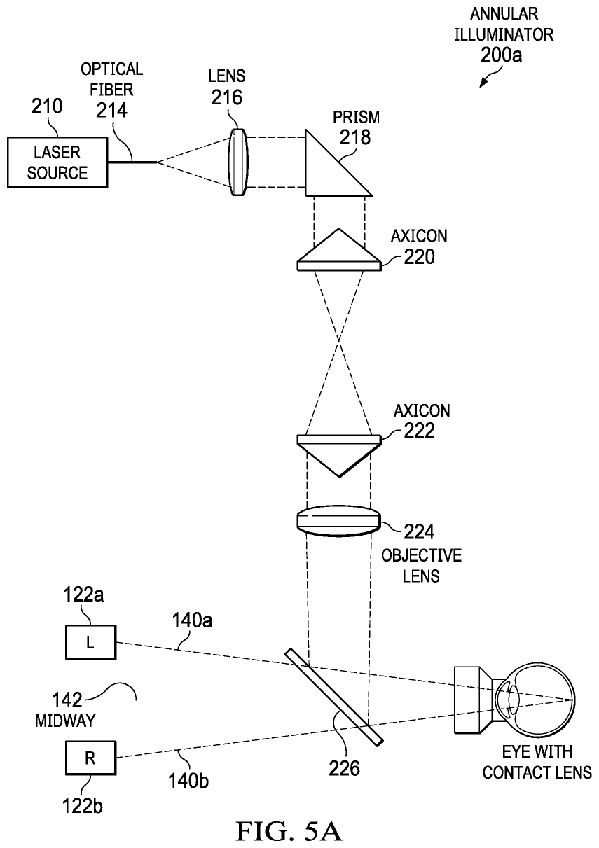

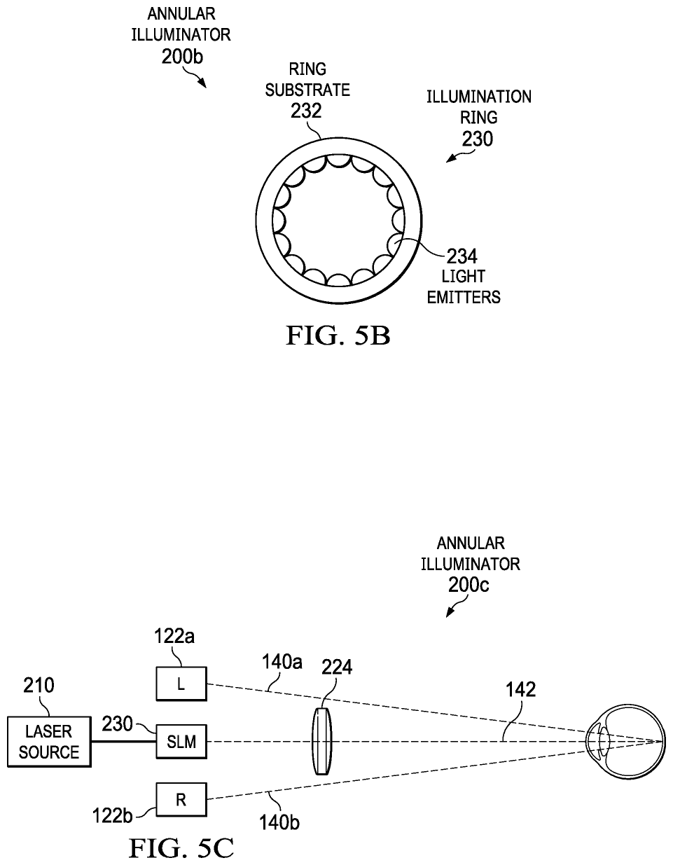

FIGS. 5A through 5C illustrate examples of annular illuminators 200 (200a, 200b, 200c) and visualization system 112, according to certain embodiments. An annular illuminator 200 directs annular illumination towards the interior of an eye. In the examples, an annular illuminator 200 comprises a light source (e.g., laser source 210) that generates illumination light and annular optical elements that modify the illumination light to yield annular illumination. In certain embodiments, the light source may generate a laser beam, e.g., a laser beam with a speckle pattern. Visualization system 112 includes oculars 122, which in turn include left L ocular 122a and right R ocular 122b, with left L view path 140a and right R view path 140b, respectively. A midway path 142 is located midway between left L view path 140a and right R view path 140b.

FIG. 5A illustrates an annular illuminator 200a with annular optical elements comprising one or more axicons. In the example, annular illuminator 200a is implemented as an angled illumination system 114b. In other examples, however, annular illuminator 200a may be implemented as a coaxial illumination system 114a, such as coaxial axicon optics on a slit lamp.

In certain embodiments, the annular optical elements comprise one, two, or more axicons that yield the annular illumination. An axicon is a lens with a conical surface that transforms a laser beam into an annular distribution. An axicon typically has a long linear depth of focus, not a point focus. In the example, the annular optical elements include a laser source 210, an optical fiber 214, a lens 216, a prism 218, an axicon 220, an axicon 222, an objective lens 224, and a beam splitter 226, optically coupled as shown. In the embodiments, laser source 210 generates a laser beam, which optical fiber 214 delivers to lens 216. Lens 216 directs the beam to prism 218, which directs the beam to axicons 220 and 222. Axicons 220 and 222 yield the annular illumination. In the embodiments, axicon 220 transforms the illumination light into an annular distribution of light, and axicon 222 modifies the annular distribution of light to yield the annular illumination.

The annular optical elements may include other or additional optical elements. For example, the annular optical elements may include an element that collimates light prior to axicons 220 and 222. As another example, the annular optical elements may include an element that yields a Bessel beam before, between, or after axicons 220 and 222. As another example, the annular optical elements may include an axicon transforms the illumination light into an annular distribution of light, and a spherical lens modifies the annular distribution of light to yield the annular illumination.

Objective lens 224 focuses the illumination light, and may comprise, e.g., a lens such as an achromat. An achromatic lens or achromat is a lens that is designed to limit the effects of chromatic and spherical aberration. Achromatic lenses are corrected to bring two wavelengths (typically red and blue) into focus on the same plane. Beam splitter 226 directs the come of light towards the eye.

FIG. 5B illustrates an annular illuminator 200b with annular optical elements comprising an illumination ring 230. In the example, annular illuminator 200b includes a ring substrate 232 and a plurality of light emitters 234. Ring substrate 232 supports light emitters 234 and may have any suitable diameter, e.g., 10 to 20 millimeters, such as similar to the diameter of the cornea, e.g., 12 mm. Light emitters 234 emit light to yield the annular illumination. Light emitters 234 may be individual light sources, e.g., LED lights (such as white or green), or may be light outputs, e.g., the output of optical fibers delivering light from a light source. Annular illuminator 200b may be implemented as a coaxial illumination system 114a or in some cases as an angled illumination system 114b.

FIG. 5C illustrates an annular illuminator 200c with annular optical elements comprising a spatial light modulator (SLM) 230. In the example, annular illuminator 200c is implemented as a coaxial illumination system 114a. In other examples, annular illuminator 200c may be implemented as an angled illumination system 114b.

In the example, annular illuminator 200c includes a laser source 210, SLM 230, and an objective lens 224, coupled as shown. Laser source 210 generates a laser beam. SLM 230 modulates the laser beam to yield annular illumination. SLM 230 may be any suitable SLM, e.g., a reflective and/or transmissive SLM or a phase-controlled SLM, such as a phase-controlled programmable liquid crystal on silicon (LCoS or LCOS) SLM. Objective lens 224 focuses the illumination light.

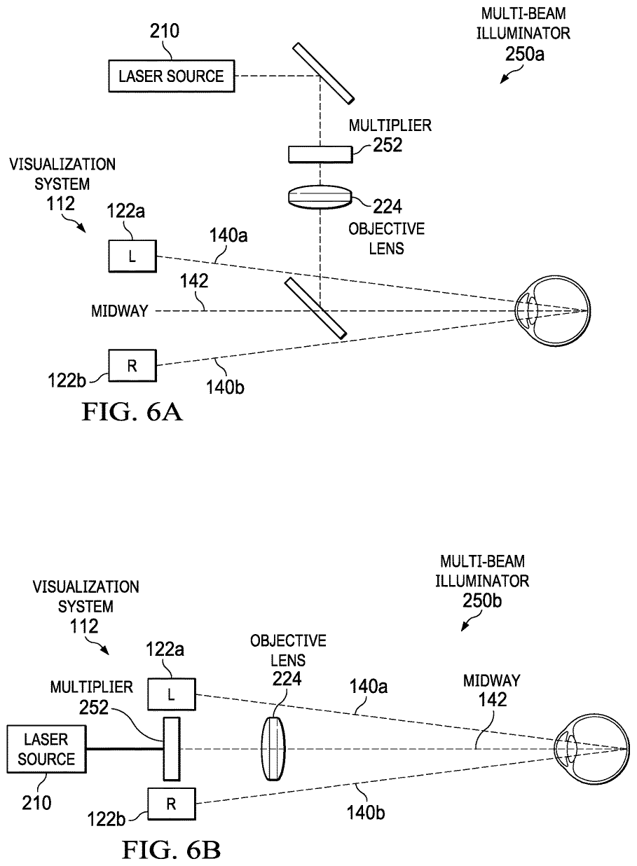

FIGS. 6A and 6B illustrate examples of multi-beam illuminators 250 and visualization system 112, according to certain embodiments. A multi-beam illuminator 250 directs a plurality of substantially parallel illumination beams towards the interior of an eye. In the examples, multi-beam illuminators 250 (250a, 250b) include a light source (e.g., a laser source 210) and multi-beam optical elements (e.g., a beam multiplier 252 and an objective lens 224), optically coupled as shown. Visualization system 112 includes oculars 122, which in turn include left L ocular 122a and right R ocular 122b, with left L view path 140a and right R view path 140b, respectively. A midway path 142 is located midway between left L view path 140a and right R view path 140b.

In certain embodiments, the light source generates illumination light. In certain embodiments, the light source may be laser source 210 that generates a laser beam, e.g., a laser beam with a speckle pattern. Multi-beam optical elements modify the illumination light to yield the illumination beams. Beam multiplier 252 modulates (e.g., multiples) the laser beam to yield the illumination beams, and objective lens 224 focuses the beams. In certain embodiments, beam multiplier 252 or other multi-beam optical element collimates the laser beam to yield substantially parallel illumination beams.

Beam multiplier 252 may comprise any suitable optical element that yields more beams from fewer beams (e.g., multiple beams from one beam), e.g., a lenslet array (e.g., a wafer optics lenslet array) or a SLM. The intersections (e.g., laser spots) of the beams with a plane orthogonal to the direction of the beams may have any suitable pattern, e.g., a rectangular or a polar array. A rectangular array comprises rows of spots, where the rows may be (but are not necessarily) equidistant from each other. The spots of the rows may or may not align into columns. A polar array comprises concentric ovals, such as concentric circles.

FIG. 6A illustrates multi-beam illuminator 250a implemented as an angled illumination system. In the example, the light output of illuminator 250a is not located on midway path 142. A beam splitter directs the illumination light onto midway path 142.

FIG. 6B illustrates multi-beam illuminator 250b implemented as a coaxial illumination system. In the example, illuminator 250b is located between left L view path 140a and right R view path 140b such that the illumination path is coincident with midway path 142.

FIG. 7 illustrates an example of a method for providing vitreoretinal visualization for ophthalmic procedures, such as a laser vitreolysis procedure, that may be performed by system 110 of FIG. 1, according to certain embodiments. The method begins at step 410, where illumination system 114 generates light. The light may be a laser beam, e.g., a laser beam with a speckle pattern.

Illumination system 114 modifies the light at step 412. Optical elements of system 114 modify the light to yield annular or multi-beam illumination. Illumination system 114 directs the light towards the interior of the eye at step 416 to illuminate at least a part or all of the vitreoretinal region, e.g., the vitreous and/or retina. The illumination may be directed coaxially or at an angle.

Visualization system 112 captures light reflected from the interior of the eye at step 418. Optical elements of visualization system 112 provide an image of the eye from the reflected light at step 420. Oculars 122 may provide the image to a user of system 110. Treatment system 116 directs a treatment laser beam towards the eye at step 422. The user may instruct treatment system 116 to direct the treatment laser beam towards a target identified in the image. The method then ends.

Certain embodiments of the optical visualization systems may have advantages over digital imaging systems. Doctors are more familiar with optical systems. In addition, digital imaging processing involves certain estimates and ambiguity that optical processes do not. Moreover, live optical systems are more reliable and require less complex software development and less regulatory approval.

A component (such as computer 118) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic system for visualizing an interior of an eye, comprising:
an illumination system configured to illuminate the interior of the eye, the illumination system comprising an annular illuminator configured to direct annular illumination towards the interior of the eye, the annular illumination having an illumination axis; and
a visualization system configured to provide an image of the interior of the eye, the visualization system comprising a plurality of visualization optical elements, the plurality of visualization optical elements comprising:
an objective lens configured to receive light reflected from the interior of the eye; and
oculars configured to transmit the reflected light to yield an image of the interior of the eye, the oculars having an ocular axis.

2. The ophthalmic system of claim 1, further comprising:
a laser device configured to direct a treatment laser beam towards the interior of the eye.

3. The ophthalmic system of claim 1, the annular illuminator comprising:
a laser source configured to provide illumination light; and
a plurality of annular optical elements configured to modify the illumination light to yield the annular illumination.

4. The ophthalmic system of claim 3, the laser source configured to provide the illumination light as a laser beam with a speckle pattern.

5. The ophthalmic system of claim 3, the plurality of annular optical elements comprising:
a first axicon configured to transform the illumination light into an annular distribution of light; and
a second axicon configured to modify the annular distribution of light to yield the annular illumination.

6. The ophthalmic system of claim 3, the plurality of annular optical elements comprising:
an axicon configured to transform the illumination light into an annular distribution of light; and
a spherical lens configured to modify the annular distribution of light to yield the annular illumination.

7. The ophthalmic system of claim 3, the plurality of annular optical elements comprising:
an achromatic lens configured to focus the annular illumination.

8. The ophthalmic system of claim 1, the annular illuminator comprising:
a laser source configured to provide an illumination light; and
a spatial light modulator configured to modify the illumination light to yield the annular illumination.

9. The ophthalmic system of claim 1, the annular illuminator comprising an illumination ring, the illumination ring comprising a plurality of lights disposed about the illumination ring.

10. The ophthalmic system of claim 1, the illumination axis substantially coincident with the ocular axis.

11. The ophthalmic system of claim 1, the illumination axis at an angle to the ocular axis.

12. The ophthalmic system of claim 1, further comprising a slit lamp microscope.

13. An ophthalmic system for visualizing an interior of an eye, comprising:
an illumination system configured to illuminate the interior of the eye, the illumination system comprising a multi-beam illuminator configured to direct a plurality of illumination beams towards the interior of the eye, the plurality of illumination beams having an illumination axis, the multi-beam illuminator comprising:
a laser source configured to provide illumination light; and
one or more multi-beam optical elements configured to modify the illumination light to yield the plurality of illumination beams; and
a visualization system configured to provide an image of the interior of the eye, the visualization system comprising a plurality of visualization optical elements, the plurality of visualization optical elements comprising:
an objective lens configured to receive light reflected from the interior of the eye; and
oculars configured to transmit the reflected light to yield an image of the interior of the eye, the oculars having an ocular axis.

14. The ophthalmic system of claim 13, further comprising:
a laser device configured to direct a treatment laser beam towards the interior of the eye.

15. The ophthalmic system of claim 13, the one or more multi-beam optical elements comprising:
a lenslet array configured to transform the illumination light into the plurality of illumination beams.

16. The ophthalmic system of claim 15, the plurality of illumination beams arranged into a circular pattern.

17. The ophthalmic system of claim 15, the plurality of illumination beams arranged into a rectangular pattern.

18. The ophthalmic system of claim 13, the illumination axis substantially coincident with the ocular axis.

19. The ophthalmic system of claim 13, the illumination axis at an angle to the ocular axis.

20. The ophthalmic system of claim 13, the laser source configured to provide a laser beam with a speckle pattern.

21. The ophthalmic system of claim 13, further comprising a slit lamp microscope.

22. An ophthalmic system for visualizing an interior of an eye, comprising:
an illumination system configured to illuminate the interior of the eye, the illumination system comprising an annular illuminator configured to direct annular illumination towards the interior of the eye, the annular illumination having an illumination axis;
a visualization system configured to provide an image of the interior of the eye, the visualization system comprising a slit lamp microscope, the slit lamp microscope comprising a plurality of visualization optical elements, the plurality of visualization optical elements comprising:
an objective lens configured to receive light reflected from the interior of the eye; and
oculars configured to transmit the reflected light to yield an image of the interior of the eye, the oculars having an ocular axis; and
a laser device configured to direct a treatment laser beam towards the interior of the eye, the annular illuminator comprising:
a laser source configured to provide illumination light with a speckle pattern and a plurality of annular optical elements configured to modify the illumination light to yield the annular illumination, the optical elements comprising: a first axicon configured to transform the illumination light into an annular distribution of light, a second axicon or a spherical lens configured to modify the annular distribution of light to yield the annular illumination, and an achromatic lens configured to focus the annular illumination; or
a laser source configured to provide an illumination light and a spatial light modulator configured to modify the illumination light to yield the annular illumination; or
an illumination ring comprising a plurality of lights disposed about the illumination ring.

23. An ophthalmic system for visualizing an interior of an eye, comprising:
an illumination system configured to illuminate the interior of the eye, the illumination system comprising a multi-beam illuminator configured to direct a plurality of illumination beams towards the interior of the eye, the plurality of illumination beams having an illumination axis, the multi-beam illuminator comprising:
a laser source configured to provide illumination light with a speckle pattern; and
one or more multi-beam optical elements configured to modify the illumination light to yield the plurality of illumination beams, the one or more multi-beam optical elements comprising a lenslet array configured to transform the illumination light into the plurality of illumination beams, the plurality of illumination beams arranged into a circular pattern or a rectangular pattern;
a visualization system configured to provide an image of the interior of the eye, the visualization system comprising a slit lamp microscope, the slit lamp microscope comprising a plurality of visualization optical elements, the plurality of visualization optical elements comprising:
an objective lens configured to receive light reflected from the interior of the eye; and
oculars configured to transmit the reflected light to yield an image of the interior of the eye, the oculars having an ocular axis; and
a laser device configured to direct a treatment laser beam towards the interior of the eye.
